(19) United States
(12) Patent Application Publication
(10) Pub. No.: US 2022/0296812 A1
(43) Pub. Date: Sep. 22, 2022

(54) INJECTOR WITH ELASTOMERIC DRIVE UNIT

(71) Applicant: WEST PHARMACEUTICAL SERVICES, INC., Exton, PA (US)

(72) Inventor: James G. GUTHLEIN, Malvern, PA (US)

(21) Appl. No.: 17/700,948

(22) Filed: Mar. 22, 2022

Related U.S. Application Data
(60) Provisional application No. 63/164,029, filed on Mar. 22, 2021.

(51) Int. Cl. A61M 5/20; A61M 5/315

(52) U.S. Cl. CPC A61M 5/20; A61M 5/31513; A61M 2205/0216; A61M 2205/8281; A61M 2005/2086; A61M 2005/3115

(57) ABSTRACT

A drive unit for an injector includes an elastomeric drive member configured to possess a first elastic potential energy in an elastically stretched state that is releasable to convert the elastic energy to a first biasing force. A selectively releasable plunger rod has a locked position maintaining the elastomeric drive member in the elastically stretched state. A trigger member is biased by a second biasing force into an interlocking position. In the interlocking position, an arm of the trigger member interlocks with a notch of the plunger rod, thereby maintaining the plunger rod in the locked position thereof. The trigger member is selectively movable against the second biasing force to an unlocking position spaced from the notch, thereby releasing the first elastic potential energy into the first biasing force to axially advance the plunger rod and thus advance the piston through the reservoir.

### CROSS REFERENCE TO RELATED APPLICATIONS

This application claims the benefit of priority to U.S. Provisional Patent Application No. 63/164,029, filed Mar. 22, 2021, the disclosure of which is hereby incorporated by reference herein.

### FIELD OF THE INVENTION

The present disclosure generally relates to drive units for injectors, and, more particularly, to elastic energy driven drive units.

### BACKGROUND

Autoinjectors allow the automated delivery of a predetermined dose of a medicament to a patient. A drive unit is a standard component of an autoinjector. Generally, when an autoinjector is activated, the drive unit operates to expel the medicament out of a reservoir of the injector for delivery to a patient, e.g., via a fluidly connected injection needle. Accordingly, drive units are essential operative components of autoinjectors. However, conventional drive units are often electrically, chemically, or electromechanically operated, and involve a complex interrelation of components. For instance, many autoinjectors typically utilize syringe type drug containers having a plunger rod, and a pre-loaded spring is often utilized as a power source for the drive unit. When triggered, the spring accelerates the syringe forward and actuates the piston of the syringe. This causes the syringe to puncture the injection site and also inject the medicament from the drug container.

Other known types of autoinjectors include an electrically powered drive unit that may be particularly suitable for use by patients whose manual dexterity is compromised, e.g., due to severe arthritis or injury. While the use of electrically powered drive systems can provide practical assistance to such patients, they also have more complex drive arrangements which are often susceptible to higher failure rates. Further, reliance on an electrical power source, such as a battery, increases the risk that the autoinjector would be unusable, e.g., in the event the battery is drained. Moreover, such electrically powered autoinjectors are not usually fully disposable, and thus provide greater risk of contamination to patients who re-use certain component parts.

Additionally, each of the conventional drive units described above are expensive to manufacture, while also being subject to overall failure in the event of any single component failure. It would, therefore, be advantageous to manufacture a mechanically operated drive unit, having reduced components while relying on elastic potential energy as a drive source, resulting in a cheaper, more reliable drive unit. Accordingly, there is a clear and substantial need for an elastomeric drive unit that solves these aforementioned problems of conventional autoinjector drive units. Another goal is to reduce the number of components needed to generate the required force for actuating the drug delivery device. Another goal is to provide a resettable drive unit for the drug delivery device. A further goal is to provide a means to stop the drug delivery process when the drug delivery device is prematurely removed from the patient, while also providing an indication of how much medicament was delivered at the time the delivery process was interrupted.

### SUMMARY

The foregoing needs are met, to a great extent, by an injector including a reservoir containing a substance; a piston sealing an end of the reservoir; an injection needle in fluid communication with the reservoir; and a drive unit. The drive unit includes an elastomeric drive member configured to possess a first elastic potential energy in an elastically stretched state. The elastomeric drive member is selectively releasable from the elastically stretched state such that the first elastic potential energy converts to a first biasing force. A plunger rod is operatively connected with the elastomeric drive member and defines a notch. The plunger rod has a locked position maintaining the elastomeric drive member in the elastically stretched state and is selectively releasable from the locked position. A trigger member. having an arm, is biased by a second biasing force into an interlocking position. In the interlocking position, the arm of the trigger member interlocks with the notch of the plunger rod, thereby maintaining the plunger rod in the locked position thereof and preventing release of the first elastic potential energy. The trigger member is selectively movable against the second biasing force to an unlocking position spaced from the notch, and, in turn, releases the first elastic potential energy into the first biasing force to axially advance the plunger rod to advance the piston through the reservoir.

According to another aspect, the body of the trigger member further includes an aperture defining a pivot axis, and a leg extending from the body, the leg being selectively engageable to pivot the trigger member from the interlocking position to the unlocking position against the second biasing force.

According to another aspect, the leg defines a greater span relative to the aperture than a span of the arm relative to the aperture.

According to another aspect, the elastomeric drive member comprises a first elastomeric band.

According to another aspect, the elastomeric drive member further comprises a second elastomeric band configured to possess a second elastic potential energy releasable into the second biasing force, the trigger member being connected with the second elastomeric band, and the second elastic potential energy of the second elastomeric band being releasable to apply the second biasing force onto the trigger member to maintain the trigger member in the interlocking position, and wherein the elastomeric drive member further comprises a platform connecting the first elastomeric band and the second elastomeric band.

According to another aspect, the trigger member further comprises a seat for receiving the second elastomeric band, whereby movement of the trigger member from the interlocking position to the unlocking position stretches the second elastomeric band and generates the second elastic potential energy.

According to another aspect, the injector may comprise a spring operable to generate the second biasing force, wherein the trigger member is movable from the interlocking position to the unlocking position against the second biasing force of the spring.

According to another aspect, the plunger rod includes a plurality of successive ratchet notches, whereby the second biasing force is operable to return the arm of the trigger member to interlock with an opposing one of the plurality of successive ratchet notches upon cessation of the selective movement of the trigger member against the second biasing force.

According to another aspect, the trigger member is a first trigger member and the notch of the plunger rod is a locking notch, the plunger rod further including a plurality of successive progression ratchet notches, and the drive unit further comprising a second trigger member opposing the plurality of successive progression ratchet notches; and a third elastomeric band configured to generate a third elastic potential energy releasable into a third biasing force, the second trigger member being connected with the third elastomeric band, whereby cessation of the selective movement of the first trigger member against the second biasing force triggers release of the third elastic potential energy into the third biasing force to interlock an arm of the second trigger member with an opposing one of the plurality of successive progression ratchet notches.

According to another aspect, the drive unit may comprise a drive unit base body, the elastomeric drive member being axially and rotatably secured to the drive unit base body, and the trigger member being axially secured to the drive unit base body and rotatable relative to the drive unit base body.

According to another aspect, the drive unit further comprises a drive unit base body having a seat configured to securely receive the platform of the elastomeric drive member; and a first post projecting from the drive unit base body and into the aperture of the trigger member to enable pivoting of the trigger member between the interlocking position and the unlocking position about the first post.

According to another aspect, the injector may comprise a second post projecting from the drive unit base body and positioned in a pivot path of the trigger member to limit a maximum pivot angle of the trigger member.

According to another aspect, the drive unit base body includes a base plate, the seat being formed along the base plate and the first post projecting from the base plate.

According to another aspect, the elastomeric drive member further comprises an elastomeric damper blade configured to contact the plunger rod and generate a drag frictional force on the plunger rod to limit a rate of axial advancement of the plunger rod upon selective movement of the trigger member into the unlocking position.

According to another aspect, the injector may further comprise a stationary dampening body configured to contact the plunger rod and generate a drag frictional force on the plunger rod to limit a rate of axial advancement of the plunger rod upon selective movement of the trigger member into the unlocking position.

According to another aspect, the elastomeric drive member may be injection molded as a single component.

According to another aspect, the injector may comprise a first housing portion housing the reservoir, the piston, and the injection needle, and a second housing portion housing the drive unit, the first housing portion and the second housing portion being selectively engageable and selectively detachable.

According to another aspect, the first housing portion is a disposable portion, and the second housing portion is a reusable portion.

According to another aspect, the plunger rod is axially retractable to generate the first elastic potential energy of the elastomeric drive member.

According to another aspect, the drive unit further comprises an actuation member configured to move the trigger member from the interlocking position to the unlocking position upon selective actuation of the actuation member by a user.

There has thus been outlined certain aspects of the present disclosure in order that the detailed description thereof herein may be better understood, and in order that the present contribution to the art may be better appreciated. There are additional aspects of the present disclosure that will be described below and which form the subject matter of the claims appended hereto. In this respect, before explaining at least one aspect of the present injector drive unit in detail, it is to be understood that the injector drive unit is not limited in its application to the details of construction and to the arrangements of the components set forth in the following description or illustrated in the drawings. The injector drive unit is capable of aspects in addition to those described, and of being practiced and carried out in various ways.

### BRIEF DESCRIPTION OF THE DRAWINGS

The following description of the disclosure will be better understood when read in conjunction with the appended drawings. It should be understood, however, that the disclosure is not limited to the precise arrangements and instrumentalities shown. In order that the present disclosure may be readily understood, aspects of the injector drive units are illustrated by way of non-limiting examples in the accompanying drawings, in which like parts are referred to with like reference numerals throughout.

FIG. 1 is a schematic view of an injector housing a drive unit in accordance with the present disclosure.

FIG. 2 is a top plan view of a drive unit in accordance with a first embodiment, with a trigger member thereof in an interlocking position.

FIG. 3 is a top and side perspective view of the drive unit of FIG. 2, with the trigger member in the interlocking position.

FIG. 4 is a top plan view of the drive unit of FIG. 2, with the trigger member in an unlocking position and a plunger rod thereof axially advanced.

FIG. 5A is a schematic top plan view of the drive unit of FIG. 2, with a handle of an injector in an original state.

FIG. 5B is a schematic top plan view of the drive unit of FIG. 2, with a handle of an injector in an actuated state.

FIG. 5C is a schematic top plan view of the drive unit of FIG. 2, with an actuation button of an injector in an original state.

FIG. 5D is a schematic top plan view of the drive unit of FIG. 2, with an actuation button of an injector in an engaged state.

FIG. 6 is a partial, expanded, top plan view of the trigger member and a second elastic band of the drive unit of FIG. 2.

FIG. 7 is a top plan view of the drive unit of FIG. 2 in a configuration wherein a locking notch thereof is a first of a plurality of successive ratchet notches.

FIG. 8 is a bottom and side perspective view of the drive unit of FIG. 2, with a base body of the drive unit removed, in an alternative configuration having a plurality of progression ratchet notches positioned separately from the locking notch.

FIG. 9A is a front elevational view of the elastomeric drive member of the drive unit of FIG. 2.

FIG. 9B is a cross-sectional view of the elastomeric drive member of the drive unit of FIG. 2, taken along sectional line 9B-9B of FIG. 9A.

FIG. 10A is a schematic, top plan view of a plunger rod of the drive unit of FIG. 2, engaged by a symmetrical damper in the form of a boss.

FIG. 10B is a schematic, top plan view of an alternative configuration of the plunger rod of the drive unit of FIG. 2, having a variably dimensioned slot, engaged by a symmetrical damper in the form of a boss.

FIG. 10C is a schematic, top plan view of the plunger rod of the drive unit of FIG. 2, engaged by an alternative configuration of a symmetrical damper.

FIG. 10D is a schematic, top plan view of the plunger rod of the drive unit of FIG. 2, engaged by a directional damper.

FIG. 11A is a partial schematic, top plan view of a drive unit in accordance with a second embodiment, with the trigger member thereof in the interlocked position.

FIG. 11B is a partial schematic, top plan view of the drive unit of FIG. 11A, with the trigger member thereof in the unlocking position.

### DETAILED DESCRIPTION

The present disclosure is directed generally to elastomeric drive units for drug delivery injectors. Certain terminology is used in the following description for convenience only and is not limiting. The words “lower,” “bottom,” “upper” and “top” designate directions in the drawings to which reference is made. The words “inwardly,” “outwardly,” “upwardly” and “downwardly” refer to directions toward and away from, respectively, the geometric center of the tray, and designated parts thereof, in accordance with the present disclosure. In describing the tray, the term proximal is used in relation to the upper end of the device and the term distal is used in relation to the bottom end of the device. Unless specifically set forth herein, the terms “a,” “an” and “the” are not limited to one element, but instead should be read as meaning “at least one.” The terminology includes the words noted above, derivatives thereof and words of similar import.

It should also be understood that the terms “about,” “approximately,” “generally,” “substantially” and like terms, used herein when referring to a dimension or characteristic of a component of the disclosure, indicate that the described dimension/characteristic is not a strict boundary or parameter and does not exclude minor variations therefrom that are functionally similar. At a minimum, such references that include a numerical parameter would include variations that, using mathematical and industrial principles accepted in the art (e.g., rounding, measurement or other systematic errors, manufacturing tolerances, etc.), would not vary the least significant digit.

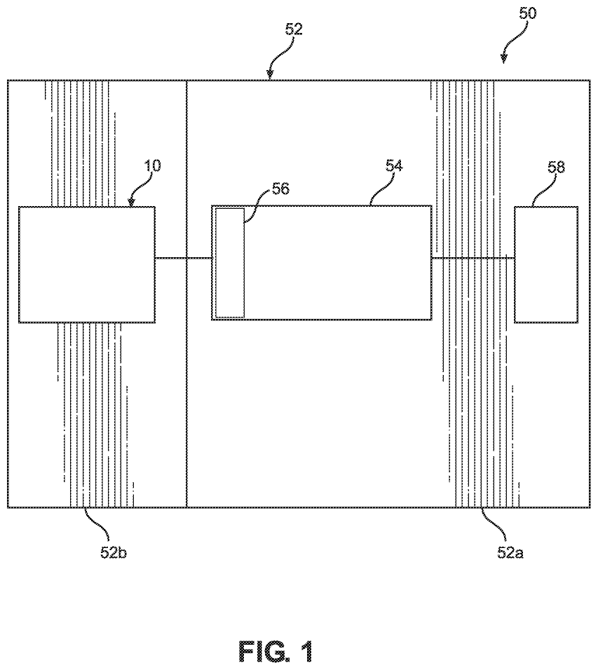

FIG. 1 illustrates an injector 50, such as an autoinjector, a pen injector, or a wearable injector, among others. The injector 50 includes an injector body 52 housing a reservoir 54 containing a substance (e.g., a medicament or other pharmaceutical substance), a piston 56 sealing an end of the reservoir 54, and an injection needle 58 in fluid communication with the reservoir 54. During use, the injector 50 is positioned upon a patient in a manner permitting the injection needle 58 to penetrate the skin surface of the patient in order to dispense the substance within the reservoir 54 into the patient. As will be described in further detail below, a drive unit 10 is configured to selectively advance the piston 56 through the reservoir 54 to dispense the substance within the reservoir 54 through the injection needle 58. The injector 50 may include a first housing portion 52a that houses the reservoir 54 and the piston 56 as well as the injection needle 58, and a second housing portion 52b that houses the drive unit 10 as well as a selective actuation member, such as a button or handle. The first housing portion 52a and the second housing portion 52b may be selectively engageable and detachable from each other.

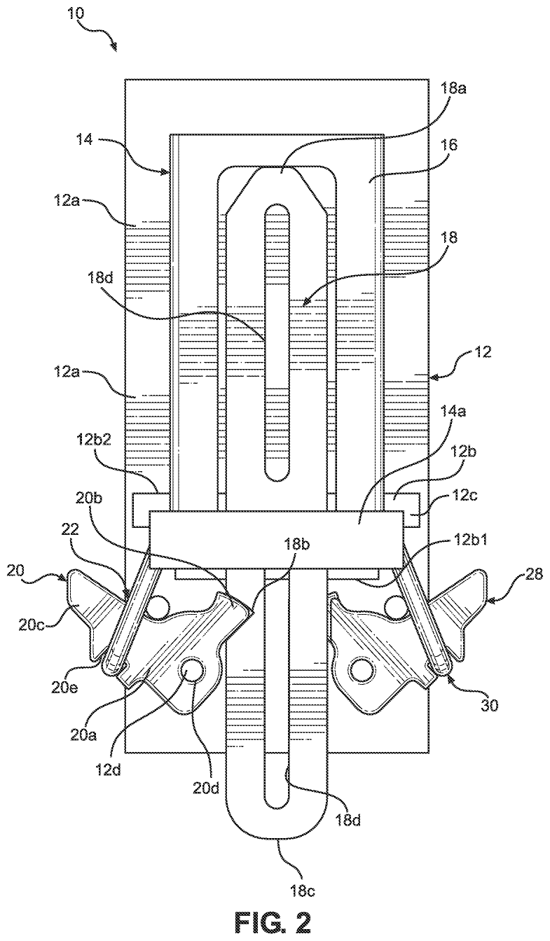

Turning to FIGS. 2-10D, the drive unit 10 includes a base body 12 defining a base plate 12a which operates as the structural backbone of the drive unit 10. The base body 12 is secured within the injector body 52 in a suitable manner (e.g., integrally, securely, releasably or the like), thereby securing the components of the drive unit 10 within the injector body 52 as necessitated. According to one implementation, for example, the base body 12 may be constructed via injection molding of a polymer, a thermoplastic polymer such as Acrylonitrile Butadiene Styrene, a polymer cement, or a combination thereof. An elastomeric drive member 14 may be axially and rotatably secured to the base body 12. That is, the elastomeric drive member 14 as a whole is not translatable or rotatable relative to the base body 12. Stated another way, the elastomeric drive member 14 is fixedly secured to the base body. According to one implementation, for example, the elastomeric drive member 14 may be constructed via injection molding, in whole or in part, of a silicone rubber, a nitrile, a thermoplastic elastomer such as Hytrel® owned by DuPont Polymers, Inc. or Santoprene® owned by Exxon Mobil Corporation, or combinations thereof.

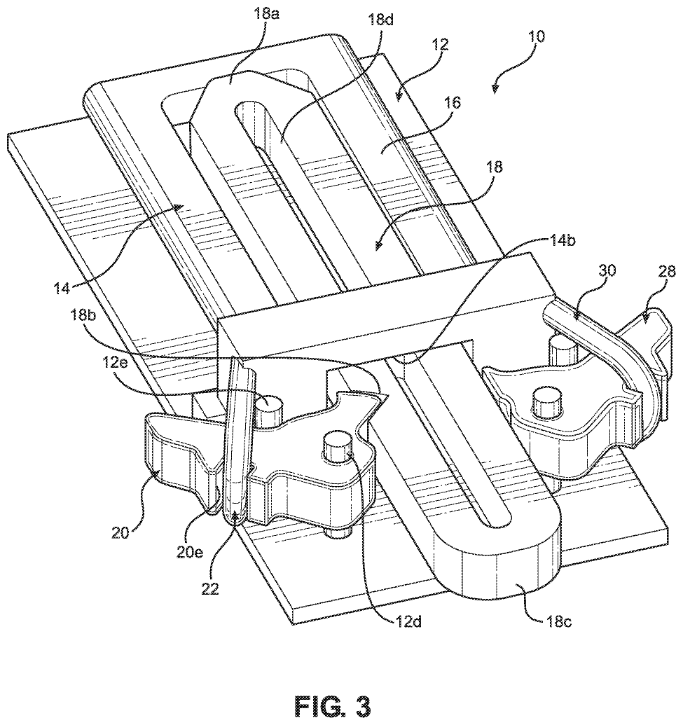

As shown in FIGS. 2 and 3, the elastomeric drive member 14 includes a platform 14a securely received within a correspondingly sized fixture seat 12b upon the base plate 12a. The fixture seat 12b includes a front wall 12b1 and a rear wall 12b2. The front and rear walls 12b1, 12b2 each project from the base plate 12a and are positioned to secure the front and back of the platform 14a therebetween to prevent axial movement of the platform 14a, as well as to prevent rotation of the platform 14a. The rear wall 12b2 includes forwardly extending flanged ends 12c positioned to secure the left and right sides of the platform 14a therebetween to prevent lateral movement of the platform 14a. According to some aspects, the elastomeric drive member 14 may be additionally or alternatively axially and rotatably secured to the base body 12 via various other methods. For example, without limitation, the platform 14a may be inserted within a correspondingly sized crevice etched into the base body 12, adhesively bonded to the base body 12, otherwise fastened to the base body 12, or a combination thereof.

As shown in FIGS. 2-8, the elastomeric drive member 14 includes a first elastomeric band 16 axially extending rearwardly from the platform 14a. The first elastomeric band 16 is stretchable into an elastically stretched state (as depicted in FIGS. 2, 3, 5A, 5C, 7, and 8), wherein the first elastomeric band 16 possesses a first elastic potential energy, as will be described further below. The first elastomeric band 16 may be comprised of natural rubber, silicone rubber, polyurethane, or polybutadiene, among other elastomers exhibiting elastic or rubber-like properties. The first elastomeric band 16 is also selectively releasable from the elastically stretched state thereof, such that the first elastic potential energy is converted/translated into kinetic energy in the form of a first biasing force, as also will be described further below. An elongate plunger rod 18 is operatively connected with the first elastomeric band 16. According to one implementation, for example, the plunger rod 18 may be constructed via injection molding of a polymer, a thermoplastic polymer such as Acrylonitrile Butadiene Styrene, a polymer cement, or a combination thereof. As shown, the plunger rod 18 axially extends substantially parallel to the base plate 12a and is axially translatable relative thereto.

In particular, the plunger rod 18 extends through an aperture 14b in the platform 14a and is axially slidable therethrough and relative to the platform 14a and the base plate 12a. A portion of the plunger rod 18, including a rear end 18a of the plunger rod 18, is enveloped by the first elastomeric band 16. Stated another way, the first elastomeric band 16 operates as a slingshot or catapult, and the plunger rod 18 operates as the corresponding projectile object placed within the slingshot or catapult. Accordingly, the plunger rod 18 increasingly stretches or draws the first elastomeric band 16 with increased rearward extension of the plunger rod 18 relative to the platform 14a, thereby generating and storing the first elastic potential energy in the first elastomeric band 16. A front end 18c of the plunger rod 18 is configured to engage with the piston 56 of the reservoir 54 of the injector 50. According to some aspects, the plunger rod 18 may also include at least one axially elongate slot or channel 18d extending therethrough and configured to receive a damper, such as a boss projecting from the base plate 12a and into engagement with the plunger rod 18 to provide a drag frictional force during axial movement of the plunger rod, as will be discussed in greater detail below.

The drive unit 10 further includes a first trigger member 20 configured to maintain the plunger rod 18 in a stationary state, such as a locked position of the plunger rod 18 depicted in FIGS. 2, 3, 5A, and 5C. The first trigger member 20 is selectively manipulable to disengage from the plunger rod 18 in order to allow the plunger rod 18 to move, as will be described in further detail. In one implementation, for example, the first trigger member 20 may be constructed via injection molding, in whole or in part, of a polymer, such as Delrin® owned by DuPont Polymers, Inc. or the like.

As shown in FIGS. 2-8, the first trigger member 20 includes a body 20a having an arm 20b extending therefrom. The first trigger member 20 also includes a leg 20c extending therefrom and angularly spaced relative to the arm 20b. According to some aspects, the arm 20b and the leg 20c may define an approximately 90° included angle therebetween. The base body 12 includes a post 12d projecting substantially perpendicularly from the base plate 12a and through an aperture 20d in the body 20a of the first trigger member 20, thereby mounting the first trigger member 20 to the base body 12 in a manner axially securing the first trigger member 20 to the base body 12, while also permitting rotation of the first trigger member 20 relative to the base body 12 about a pivot axis defined by the post 12d and the aperture 20d.

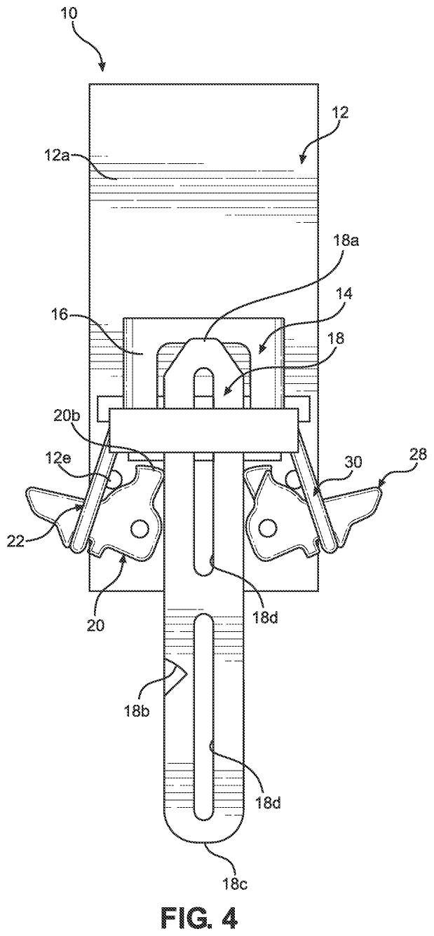

The first trigger member 20 is rotatable between an interlocking position (shown in FIGS. 2, 3, 5A, and 5C) and an unlocking position (shown in FIGS. 4, 5B, and 5D-8). When the first trigger member 20 is in the interlocking position, the arm 20b interlocks with a locking notch 18b in the plunger rod 18, thereby obstructing the plunger rod 18 from moving, i.e., maintaining the plunger rod 18 in the locked position, thereby maintaining the first elastomeric band 16 in the elastically stretched state and preventing release of the first elastic potential energy thereof. When the first trigger member 20 is in the unlocking position, the arm 20b is spaced away from the locking notch 18b, and therefore movement of the plunger rod is no longer obstructed by the arm 20b.

The elastomeric drive member 14 further includes a second elastomeric band 22 configured to bias the first trigger member 20 into the interlocking position. In one implementation, the second elastomeric band 22 may be constructed of the same material as the first elastomeric band 16. For instance, the second elastomeric band 22 may comprise natural rubber, silicone rubber, polyurethane, or polybutadiene, among other elastomers exhibiting elastic or rubber-like properties. According to some aspects, the second elastomeric band 22 may be monolithically formed with the platform 14a and the first elastomeric band 16, e.g., via injection molding as a single component, or otherwise integrally formed with one another. Similar to the first elastomeric band 16, the second elastomeric band 22 possesses and stores a second elastic potential energy when stretched, which is converted/translated into kinetic energy in the form of a second biasing force when released.

As shown in FIGS. 2 and 3, the first trigger member 20 includes a seat 20e located in a periphery of the leg 20c. The second elastomeric band 22 extends forwardly from the platform 14a such that a front end of the second elastomeric band 22 is received in the seat 20e. In a non-manipulated state of the first trigger member 20, the second elastomeric band 22 is maintained in a released state. As such, the second elastomeric band 22 is not obstructed from applying the second biasing force to the first trigger member 20, thereby urging the first trigger member 20 into the interlocking position. Stated another way, the second elastomeric band 22 applies the second biasing force onto the first trigger member 20, thereby biasing and maintaining the first trigger member 20 in the interlocked position. More particularly, the first trigger member 20 rotates in a first direction about the aperture 20d secured to the post 12d of the base body 12 as the second elastomeric band 22 applies the second biasing force to the first trigger member 20, such that the arm 20b of the first trigger member 20 moves into interlocking engagement with the locking notch 18b of the plunger rod 18. Accordingly, the second elastomeric band 22 is operable to automatically lock the first trigger member 20 with the plunger rod 18.

To release the plunger rod 18 from the locked position, the first trigger member 20 is selectively moved against the bias of the second biasing force of the second elastomeric band 22, such that the first trigger member 20 rotates in a second direction about the aperture 20d secured to the post 12d of the base body 12, wherein the second direction or rotation of the first trigger member is opposite to the first direction of rotation of the first trigger member. Rotating the first trigger member 20 in the second direction thereby disengages the arm 20b of the first trigger member from the locking notch 18b of the plunger rod 18. Stated another way, the arm 20b is moved against the bias of the second biasing force from the interlocked position to the unlocked position.

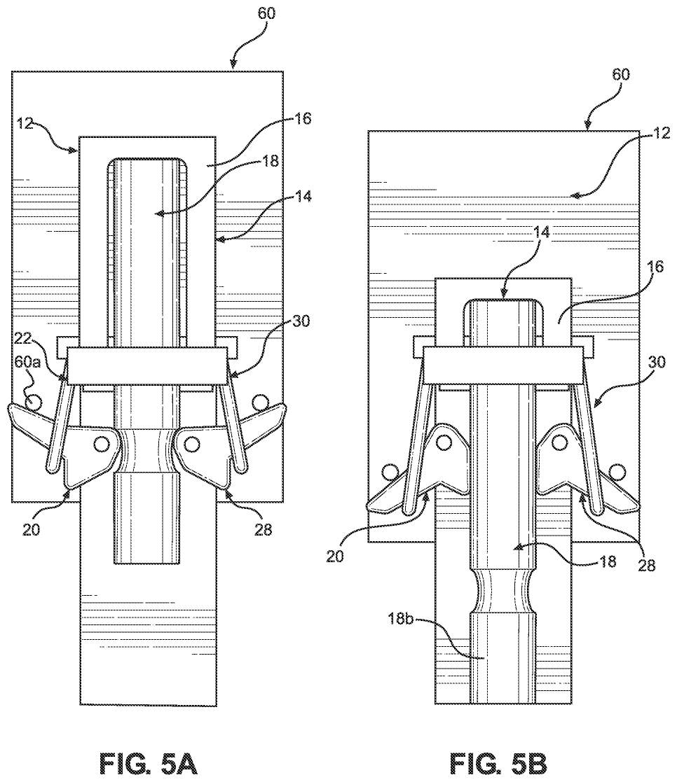

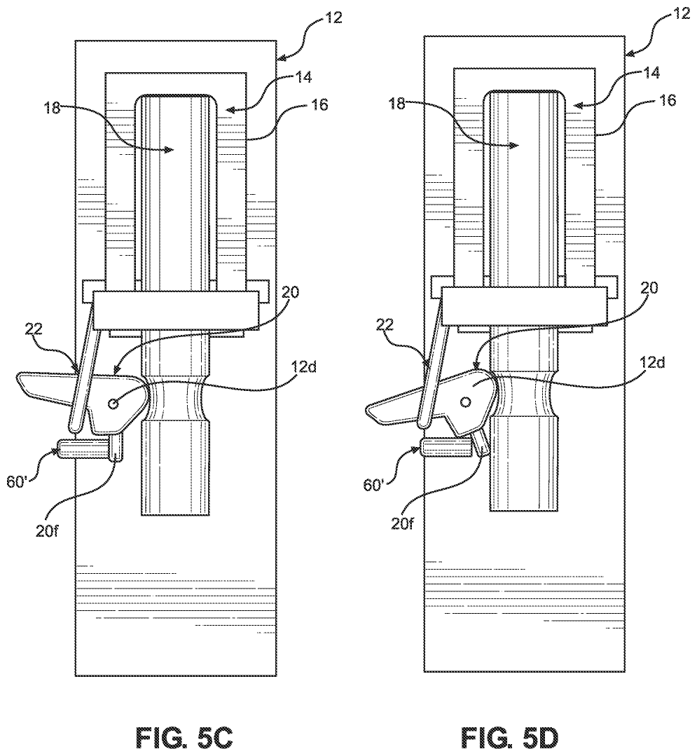

Referring to FIGS. 5A-5D, for example, the injector 50 may include a selective actuation member, such as a button or a handle, that is mechanically or otherwise operatively engageable with the first trigger member 20 to move the first trigger member 20 into the unlocking position. According to an implementation shown in FIG. 5A, the actuation member includes a handle 60 having a contact member 60a positioned to contact the leg 20c of the first trigger member 20 upon depression of the handle 60, and rotate or pivot the first trigger member 20 about the post 12d. As a result, the arm 20b disengages from the locking notch 18b of the plunger rod 18, thereby allowing the plunger rod 18 to be axially moved by the elastomeric drive member 14, as shown in FIG. 5B. According to another implementation shown in FIG. 5C, the actuation member includes a button 60′ positioned to directly contact a projection or nub 20f extending from the leg 20c of the first trigger member 20. Actuation of the button 60′ pushes the nub 20f, causing the first trigger member 20 to rotate or pivot the first trigger member 20 about the post 12d, such that the arm 20b disengages from the locking notch 18b of the plunger rod 18, as shown in FIG. 5D. In this unlocked position, the plunger rod 18 is able to be axially moved by the elastomeric drive member 14.

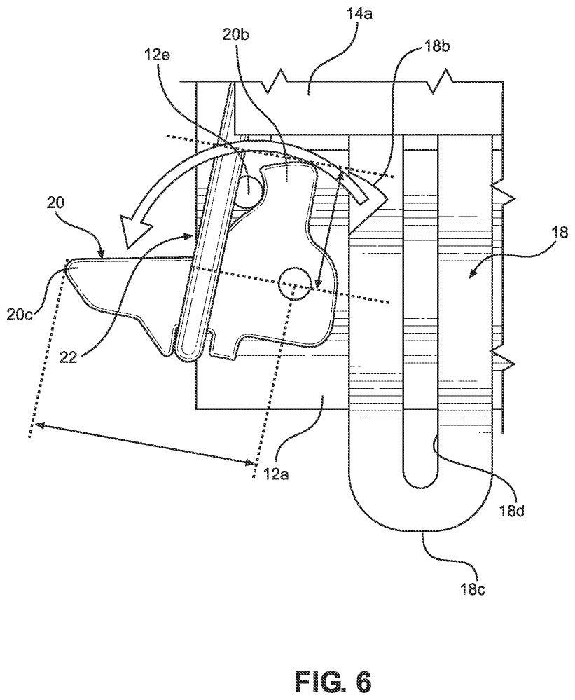

Turning to FIG. 4, the base body 12 may include a second post 12e projecting substantially perpendicularly from the base plate 12a and positioned along a pivot path of the first trigger member 20 to limit a maximum pivot angle of the first trigger member 20 when moved out of the interlocking position. Upon release of the arm 20b from the locking notch 18b, the plunger rod 18 is no longer obstructed from axial movement, and therefore, the first elastic potential energy of the first elastomeric band 16 releases in the form of the first biasing force to axially advance the plunger rod 18, and in turn, engage and/or advance the piston 56 through the reservoir 54 in order to dispense the substance therein. Furthermore, as shown in FIG. 6, the leg 20c of the first trigger member 20 may define a greater span B relative to the aperture 20d than a span A of the arm 20b relative to the aperture 20d. Accordingly, the leg 20c provides a greater moment relative to the arm 20b, thus resulting in a reduced activation force required to rotate the first trigger member 20 out of engagement with the locking notch 18b of the plunger rod 18.

For instance, in the implementation of FIGS. 5A and 5B, less activation force is required upon the handle 60 to rotate the first trigger member 20 to the unlocking position. Movement of the first trigger member 20 from the interlocking position to the unlocking position against the second biasing force of the second elastomeric band 22 stretches the second elastomeric band 22 and generates the second elastic potential energy. Maintained actuation of the button 60′ and/or maintained placement of the injector 50 upon the skin surface of the patient may be required to overcome the second biasing force and maintain the first trigger member 20 in the unlocking position. Accordingly, upon cessation of the selective movement of the first trigger member 20 into the unlocking position, the second elastic potential energy is once again released in the form of the second biasing force to rotate the first trigger member 20 back toward the interlocking position. For example, upon release of the handle 60 after initial actuation and/or removal of the injector 50 from upon the skin surface of the patient, the handle 60 subsequently returns to its original position.

Referring again to FIG. 4, the plunger rod 18 is shown having been axially advanced as a result of the application of the first biasing force thereupon via the first elastomeric band 16. In this advanced position of the plunger rod 18, the locking notch 18b is no longer aligned with the arm 20b of the first trigger member 20. Accordingly, cessation of the selective movement of the first trigger member 20 into the unlocking position results in rotation of the first trigger member 20 back toward the interlocking position under application of the second biasing force caused by the second elastomeric band, such that the arm 20b of the first trigger member 20 abuts against the plunger rod 18 without impeding the axial advancement thereof.

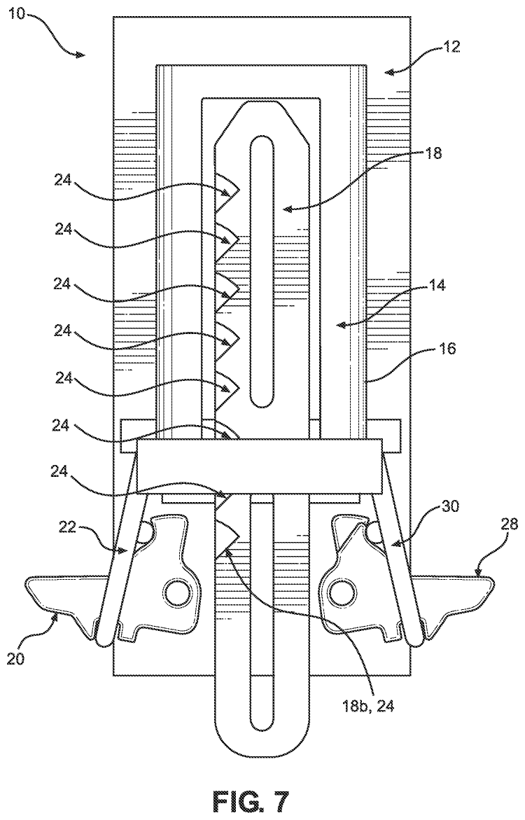

In the implementation shown in FIG. 7, the locking notch 18b is a first of a plurality of successive ratchet notches 24 located along a side of the plunger rod 18. Accordingly, cessation of the selective movement of the first trigger member 20 into the unlocking position results in rotation of the first trigger member 20 back toward the interlocking position, such that the arm 20b interlocks with one of the plurality of notches 24 aligned therewith, thereby once again obstructing the plunger rod 18 from further axial advancement. Such obstruction of the plunger rod 18 from further axial movement ceases advancement of the piston 56 through the reservoir 54, and thus stops delivery of the substance within the reservoir 54 to the patient. Accordingly, the presence of the plurality of ratchet notches 24 successively positioned along the plunger rod 18 enables the injector 50 to stop substance delivery in the event of a complication during the delivery, such as the injector 50 being prematurely removed from the patient or the actuation member being prematurely released, among other complications. In some aspects, the respective notches 24 may include gradations or markings to indicate the amount of substance dispensed upon reaching each notch 24. Thus, the cessation of substance delivery from the reservoir 54 allows for an indication of the amount of substance that was delivered to the patient at the time the delivery process was interrupted.

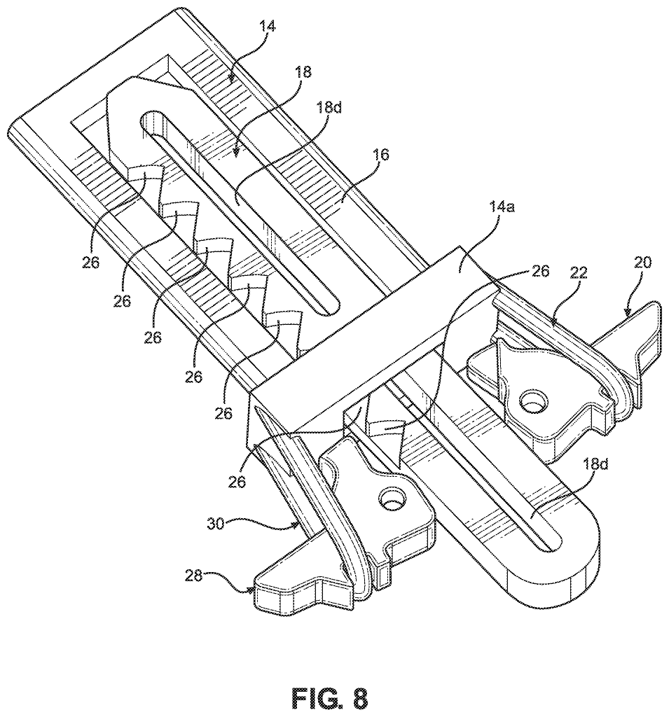

In the implementation shown in FIG. 8. the plunger rod 18 includes a plurality of progression ratchet notches 26 positioned separately from the locking notch 18b. The progression ratchet notches 26 are positioned along the plunger rod 18 on an opposite side and peripheral edge from the locking notch 18b. For example, where the locking notch 18b is located in a top surface of the plunger rod 18 along a first side periphery, the progression ratchet notches 26 are located in an underside of the plunger rod 18 along an opposing second side periphery. In such a configuration, the drive unit 10 may further include a second trigger member 28 opposing the progression ratchet notches 26, as well as a third elastomeric band 30 connected to the second trigger member 28. The second trigger member 28 is selectively manipulable to disengage from a corresponding progression ratchet notch 26 of the plunger rod 18 in order to allow the plunger rod 18 to axially advance.

In each of the implementations shown in FIGS. 2-8, the second trigger member 28 and the third elastomeric band 30 are substantially the same (e.g., shape, size, and/or material) as the first trigger member 20 and the second elastomeric band 22, respectively, and are positioned in a mirrored orientation thereto, relative to the plunger rod 18. That is, for example, the third elastomeric band 30 possesses and stores a third elastic potential energy when stretched, which is converted/translated into kinetic energy in the form of a third biasing force when released. The third elastomeric band 30 may also be monolithically formed with the platform 14a and the first elastomeric band 16, e.g., via injection molding as a single component, or otherwise integrally formed with one another. Similar to the second elastomeric band 22 described above, when the third elastomeric band 30 is in a non-manipulated state, the third biasing force is applied to the second trigger member 28, thereby biasing and maintaining the second trigger member 28 to interlock with one of the progression ratchet notches 26 aligned therewith or otherwise abut against a periphery of the plunger rod 18. Stated another way, when in the non-manipulated state, the third elastomeric band 30 is released such that it is not obstructed from applying the third biasing force to the second trigger member 28.

Selectively activating the actuation member, i.e., by depressing the button or the handle 60, may additionally rotate or pivot the second trigger member 28 against the third biasing force, and thereby move the second trigger member 28 out of engagement with the plunger rod 18. Subsequent cessation of the selective movement of the second trigger member 28 out of engagement with the progression ratchet notches 26 will result in rotation of the second trigger member 28 in a direction back toward the plunger rod 18 under the application of the third biasing force, thereby causing engagement of the second trigger member 28 with one of the progression ratchet notches 26 aligned therewith. As a result, the plunger rod 18 is obstructed from further axial advancement. Thus, employment of the progression ratchet notches 26, the second trigger member 28, and the third elastomeric band 30 serves to enable the injector 50 to stop substance delivery in the event of a complication during delivery. Moreover, the progression ratchet notches 26, the second trigger member 28, and the third elastomeric band 30 also serve to indicate how much substance was delivered to the patient at the time the delivery process was interrupted, e.g., via the inclusion of gradations or markings as previously described.

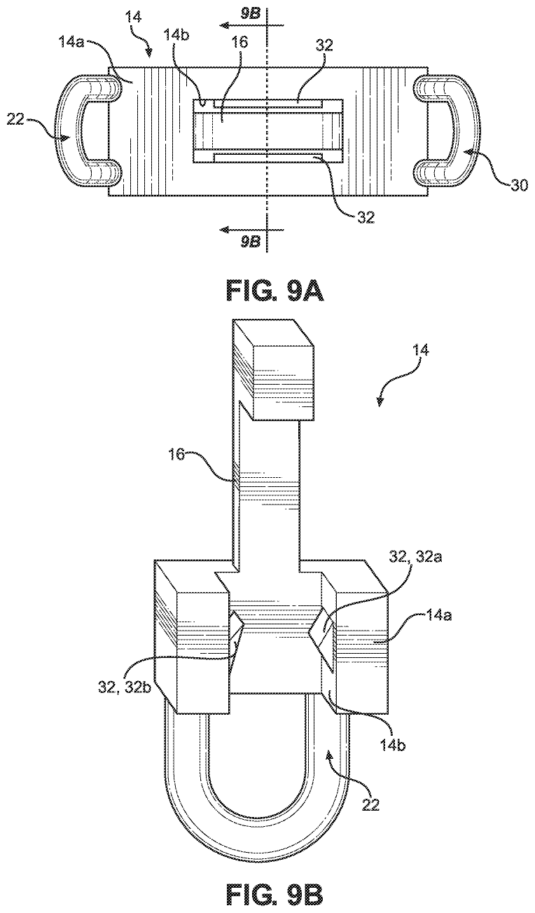

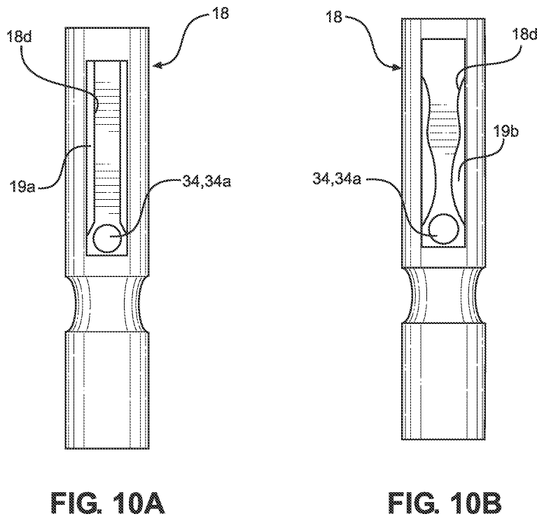

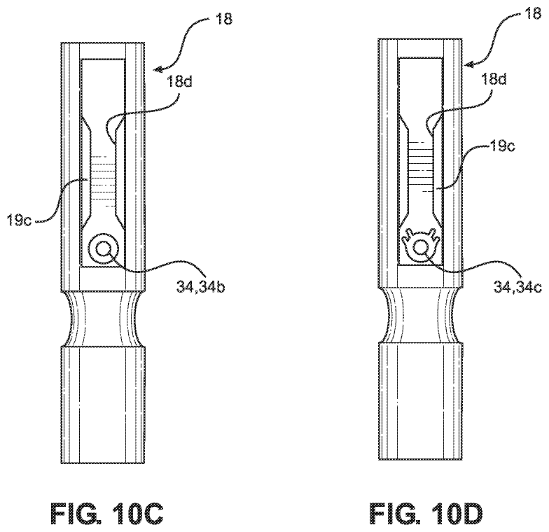

The magnitude of the first biasing force contributes to the force of advancement of the plunger rod 18, and in turn, the substance delivery rate. Accordingly, the material of the first elastomeric band 16 may be selected according to elastic energy generation properties and the desired delivery rate. Additionally, or alternatively, as shown in FIGS. 9A-10D, the drive unit 10 may also include a damper configured to contact the plunger rod 18 and generate a drag frictional force on the plunger rod 18 to limit the rate of axial advancement thereof, and in turn, adjust the delivery rate of substance from the reservoir. Similar to the selection of the first elastomeric band 16, the damper material may also be selected according to drag frictional force properties.

In some implementations, the platform 14a may include at least one elastomeric damper 32, such as a damper blade, positioned along an internal periphery of the platform 14a defining the aperture 14b. FIGS. 9A and 9B, for instance, depict a first damper blade 32a and a second damper blade 32b that respectively protrude into the aperture 14b from opposite sides of the internal periphery of the platform 14a. The first and second damper blades 32a, 32b may be constructed of the same material as the platform 14a. Each damper blade 32a, 32b projects into the space defining the aperture 14b and is dimensioned to contact the plunger rod 18 as it slides through the aperture 14b, thereby generating the drag frictional force. The damper 32 may be symmetrically shaped or non-symmetrically shaped. As shown in FIG. 9B, for instance, the first damper blade 32a may be symmetrical in shape, and therefore applies the same drag frictional force upon the plunger rod 18 during advancement or retraction of the plunger rod 18, i.e., in either sliding direction of the plunger rod 18 through the aperture 14b. Further, the second damper blade 32b may be non-symmetrically shaped to apply more drag frictional force upon the plunger rod 18 in one sliding direction thereof (e.g., axial advancement) than in the opposing sliding direction thereof (e.g., axial retraction), such that the second damper blade 32b applies almost no drag frictional force, or a negligible drag frictional force, upon the plunger rod 18 during axial retraction thereof.

As shown in FIGS. 10A-10D, the base body 12 may also include a damper in the form of a boss 34 projecting from the base plate 12a and into engagement with the plunger rod 18. The plunger rod 18 may include at least one axially elongate slot or channel 18d extending therethrough, with the boss 34 projecting therein and dimensioned to contact the periphery of the slot 18d. In the implementation shown in FIGS. 10A and 10B, for instance, the boss 34a may be symmetrical (e.g., having a cylindrical shape) and constructed of elastomeric or rigid material operable to apply the same drag frictional force in either axial direction of movement of the plunger rod 18. In the implementation shown in FIG. 10C, the boss 34b may be constructed of an elastomeric sleeve covering a rigid core. In the implementation shown in FIG. 10D, the boss 34c may be constructed of an elastomeric sleeve covering a rigid core, wherein the elastomeric sleeve is non-symmetrically shaped to apply more drag frictional force upon the plunger rod 18 in one axial direction of movement of the plunger rod 18 than the opposite axial direction of movement.

Additionally, as shown in FIG. 10A, the slot 18d may include a pair of inwardly protruding elastomeric ribs 19a disposed on opposing sides of the slot 18d. The ribs 19a define a uniformly dimensioned cross-section of the slot 18d along its axial length, thereby resulting in constant contact with the boss 34a during axial motion of the plunger rod. Such constant contact provides uniform dampening to the plunger rod 18 during axial advancement thereof. As shown in FIG. 10B, the slot 18d may include a pair of inwardly protruding elastomeric ribs 19b disposed on opposing sides of the slot 18d. The ribs 19b define a variably dimensioned cross-section of the slot 18d along its axial length, thereby resulting in different degrees of contact with the boss 34a during axial motion of the plunger rod 18, and thus providing an extent of varied dampening during axial motion of the plunger rod 18. Further, as shown in FIGS. 10C and 10D, the slot may include a pair of inwardly protruding rigid ribs 19c disposed on opposing sides of the slot 18d, wherein the rigid ribs 19c are configured to contact the elastomeric sleeve portion of the respective bosses 34b, 34c. The rigid ribs 19c extend along a portion axial length of the slot 18d that is less than the entire axial length of the slot. Accordingly, the rigid ribs 19c define a variably dimensioned cross-section along the entire axial length of the slot 18d, which results in different degrees of contact with the respective bosses 34b, 34c during axial motion of the plunger rod 18. The rigid ribs 19c thus provide varied dampening during axial motion of the plunger rod 18.

Referring back to FIG. 1, the second housing portion 52b may be a reusable portion, whereas the first housing portion 52a may be a disposable portion. For instance, upon completion of substance delivery from the reservoir 54 and injection needle 58, the injector 50 is subsequently removed from the patient's skin surface. The first and second housing portions 52a, 52b may then be detached and the first housing portion 52a disposed of. The plunger rod 18 of the drive unit 10 may then be manually axially retracted to regenerate the first elastic potential energy of the first elastomeric band 16. For instance, the actuation member may be depressed, as previously described, to disengage the first trigger member 20 (as well as the second trigger member 28 if employed) from the plunger rod 18, thereby enabling axial retraction of the plunger rod 18 against the first biasing force. Thereafter, upon sufficient axial retraction of the plunger rod 18, the actuation member may be released, so that the second elastic potential energy is once again released in the form of the second biasing force to rotate the first trigger member 20 back into the interlocking position thereof. The drive unit 10 and the second housing portion 52b are subsequently reset and ready to be re-deployed with a new first housing portion 52a. In addition to the advantage of such reusability of the drive unit 10 and the second housing portion 52b, fewer number of components are required to form the drive unit 10, thus being more cost effective than conventional drive units. The components forming the drive unit 10 also interact with each other and operate mechanically, thereby reducing the potential for failure of the drive unit 10. Therefore, the drive unit 10 of the present disclosure is a lower cost, highly reliable drive unit.

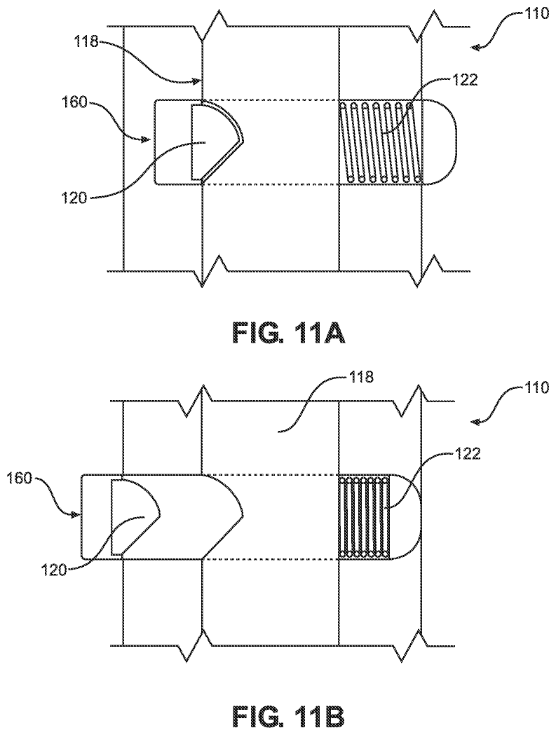

FIGS. 11A-11B illustrate another embodiment of the drive unit 110. The reference numerals of the present embodiment are distinguishable from those of the above-described embodiment by a factor of one-hundred (100), but otherwise represent the same elements as indicated above, except as otherwise specified. The drive unit 110 is substantially similar to the drive unit 10, and therefore the description of certain similarities between each drive unit may be omitted herein for the sake of brevity and convenience, and, therefore, is not limiting.

A primary difference between the drive units 10 and 110 pertains to the first trigger member 120 and the spring 122 generating the second biasing force. In the drive unit 110, the first trigger member 120 takes the form of a boss incorporated into, and projecting from, the button 160 that is laterally movable or translatable, i.e., in a direction substantially perpendicular to the axial direction of movement of the plunger rod 118. The button 160 is positioned to align with the locking notch 118b in the plunger rod 118. The button 160 is laterally translatable below (or above) the plunger rod 118 and extends beyond the plunger rod 118. On an opposite side of the plunger rod 118 from the boss 120, a spring 122, such as a helical compression spring, is also incorporated into the button 160. The spring 122 is interposed between the periphery of the plunger rod 118 and a terminal end of the button 160 that is engageable by a user.

The helical spring 122 is operable to possess and store a second elastic potential energy when compressed, which is converted or translated into kinetic energy in the form of a second biasing force when released to make the helical coils expand back toward their original length. The helical spring 122 is expanded on one side of the plunger rod 118, in an original state thereof under the application of the second biasing force, thereby engaging the boss 120 with the locking notch 118b on an opposite side of the plunger rod 118 and maintaining the boss 120 in the interlocked position, as shown in FIG. 11A. Depression of the button 160 compresses the helical spring 122 and moves the boss 120 into the unlocking position against the second biasing force of the helical spring 122, thereby freeing the plunger rod 118 to axially advance under the application of the first biasing force (in a similar manner as explained above with respect to the drive unit 10).

While certain implementations of an injector having an elastomeric drive unit, and the associated methods of use and manufacture thereof, have been described in terms of what may be considered to be specific aspects, the present disclosure is not limited to the disclosed aspects. Additional modifications and improvements to the aforementioned drive units may be apparent to those skilled in the art. Furthermore, implementations described and shown in the accompanying drawings are provided as examples of ways in which the drive unit may be put into effect and are not intended to be limiting on the scope of the disclosure. Modifications may be made, and elements may be replaced with functionally and structurally equivalent parts, and features of different embodiments may be combined without departing from the disclosure. The many features and advantages of the disclosure are apparent from the detailed specification, and thus, it is intended by the appended claims to cover all such features and advantages of the present disclosure which fall within the spirit and scope of the disclosure.

What is claimed is:

1. An injector for dispensing a substance, the injector comprising:
a reservoir containing the substance;
a piston sealing an end of the reservoir;
an injection needle in fluid communication with the reservoir; and
a drive unit configured to selectively advance the piston within the reservoir, the drive unit comprising:
an elastomeric drive member configured to possess a first elastic potential energy in an elastically stretched state, the elastomeric drive member being selectively releasable from the elastically stretched state such that the first elastic potential energy converts to a first biasing force;
a plunger rod operatively connected with the elastomeric drive member, the plunger rod defining a notch and having a locked position maintaining the elastomeric drive member in the elastically stretched state, and the plunger rod being selectively releasable from the locked position; and
a trigger member comprising a body having an arm extending therefrom, the trigger member being biased by a second biasing force into an interlocking position, and the trigger member being selectively movable against the second biasing force to an unlocking position;
wherein, in the interlocking position, the arm of the trigger member interlocks with the notch of the plunger rod, thereby maintaining the plunger rod in the locked position and preventing release of the first elastic potential energy, and
wherein, in the unlocking position, the arm of the trigger member is spaced from the notch of the plunger rod, thereby releasing the first elastic potential energy into the first biasing force to axially advance the plunger rod, and thereby advance the piston through the reservoir.

2. The injector of claim 1, wherein the body of the trigger member further includes an aperture defining a pivot axis, and a leg extending from the body, the leg being selectively engageable to pivot the trigger member from the interlocking position to the unlocking position against the second biasing force.

3. The injector of claim 2, wherein the leg defines a greater span relative to the aperture than a span of the arm relative to the aperture.

4. The injector of claim 1, wherein the elastomeric drive member comprises a first elastomeric band.

5. The injector of claim 4, wherein the elastomeric drive member further comprises a second elastomeric band configured to possess a second elastic potential energy releasable into the second biasing force, the trigger member being connected with the second elastomeric band, and the second elastic potential energy of the second elastomeric band being releasable to apply the second biasing force onto the trigger member to maintain the trigger member in the interlocking position, and wherein the elastomeric drive member further comprises a platform connecting the first elastomeric band and the second elastomeric band.

6. The injector of claim 5, wherein the trigger member further comprises a seat for receiving the second elastomeric band, whereby movement of the trigger member from the interlocking position to the unlocking position stretches the second elastomeric band and generates the second elastic potential energy.

7. The injector of claim 1, further comprising a spring operable to generate the second biasing force, wherein the trigger member is movable from the interlocking position to the unlocking position against the second biasing force of the spring.

8. The injector of claim 1, wherein the plunger rod includes a plurality of successive ratchet notches, whereby the second biasing force is operable to return the arm of the trigger member to interlock with an opposing one of the plurality of successive ratchet notches upon cessation of the selective movement of the trigger member against the second biasing force.

9. The injector of claim 1, wherein the trigger member is a first trigger member and the notch of the plunger rod is a locking notch, the plunger rod further including a plurality of successive progression ratchet notches, and the drive unit further comprising:
a second trigger member opposing the plurality of successive progression ratchet notches; and
a third elastomeric band configured to generate a third elastic potential energy releasable into a third biasing force, the second trigger member being connected with the third elastomeric band, whereby cessation of the selective movement of the first trigger member against the second biasing force triggers release of the third elastic potential energy into the third biasing force to interlock an arm of the second trigger member with an opposing one of the plurality of successive progression ratchet notches.

10. The injector of claim 1, further comprising a drive unit base body, the elastomeric drive member being axially and rotatably secured to the drive unit base body, and the trigger member being axially secured to the drive unit base body and rotatable relative to the drive unit base body.

11. The injector of claim 5, wherein the drive unit further comprises a drive unit base body having a seat configured to securely receive the platform of the elastomeric drive member; and
a first post projecting from the drive unit base body and into the aperture of the trigger member to enable pivoting of the trigger member between the interlocking position and the unlocking position about the first post.

12. The injector of claim 11, further comprising a second post projecting from the drive unit base body and positioned in a pivot path of the trigger member to limit a maximum pivot angle of the trigger member.

13. The injector of claim 11, wherein the drive unit base body includes a base plate, the seat being formed along the base plate and the first post projecting from the base plate.

14. The injector of claim 1, wherein the elastomeric drive member further comprises an elastomeric damper blade configured to contact the plunger rod and generate a drag frictional force on the plunger rod to limit a rate of axial advancement of the plunger rod upon selective movement of the trigger member into the unlocking position.

15. The injector of claim 1, further comprising a stationary dampening body configured to contact the plunger rod and generate a drag frictional force on the plunger rod to limit a rate of axial advancement of the plunger rod upon selective movement of the trigger member into the unlocking position.

16. The injector of claim 1, wherein the elastomeric drive member is injection molded as a single component.

17. The injector of claim 1, further comprising a first housing portion housing the reservoir, the piston, and the injection needle, and a second housing portion housing the drive unit, the first housing portion and the second housing portion being selectively engageable and selectively detachable.

18. The injector of claim 17, wherein the first housing portion is a disposable portion, and the second housing portion is a reusable portion.

19. The injector of claim 1, wherein the plunger rod is axially retractable to generate the first elastic potential energy of the elastomeric drive member.

20. The injector of claim 1, further comprising an actuation member configured to move the trigger member from the interlocking position to the unlocking position upon selective actuation of the actuation member by a user.
